(19) United States
(12) Patent Application Publication
(10) Pub. No.: US 2009/0120167 A1
(43) Pub. Date: May 14, 2009

(54) Device and a Method for Analysis of an Article of Food by Means of a Test Cup

(75) Inventor: Dagbjorn Skipnes, Stavanger (NO)

Correspondence Address: JAMES C. WRAY, 1493 CHAIN BRIDGE ROAD, SUITE 300, MCLEAN, VA, 22101, US

(73) Assignee: Norconserv AS

(21) Appl. No.: 11/884,715

(22) PCT Filed: Feb. 17, 2006
(86) PCT No.: PCT/NO2006/000066

§ 371 Date: Sep. 4, 2007

(30) Foreign Application Priority Data
Feb. 21, 2005 (NO) 20050910

(51) Int. Cl. G01N 33/12

(52) U.S. Cl. 73/52

(57) ABSTRACT

A test cup (1) for use in testing for example food (40) or feed in a laboratory, for example, is disclosed. The test cup (1) is comprised of a tubular body (3) arranged for internal reception of a filter (5) that divides an internal cavity of the test cup (1) into a bottom portion and a top portion, wherein the test cup (1) is provided with one of at least two dissimilar, removable and sealing bottom lids (20, 20′), and also with one of at least two dissimilar, removable and sealing top lids (15, 15′), the configuration of which is adapted for the type of measurement to be carried out. A method of using the test cup (1) is also disclosed.

The present invention relates to a test cup. More particularly, it concerns a test cup arranged for use in testing or analysing an article of food such as, but not limited to, meat from fish, birds or mammals, or feed for fish and animals The test cup is comprised of a tubular body arranged so as to be able to receive a sealing bottom lid and top lid adapted for the measuring desired to be carried out. The invention also relates to a method for practising the invention.

The object of the invention is to provide a device and a method for analysing an article of food by means of a test cup, in which the article of food may undergo a rapid heating process and one or more subsequent measurements without having to touch or remove the article of food from the test cup. Hereinafter the article of food will be referred to as “meat” or “test piece” without thereby intending to limit the range of application of the invention in any way. Also, the concept of “feed”, where relevant, is included within the concept of “food” or “foodstuff”.

Today there is an increasing market for precooked food products. In this context, precooked food products are meant to be products that have been completely or partially processed for the consumer, in which the food only requires a simple and quick preparation, for example heating.

In connection with industrial heat treatment of an article of food such as, but not limited to, fish meat, very rapid heating is desirable, whereby the article of food reaches the desired core temperature in a short time, thereby reducing the strain on the outside of, for example, the fish meat as much as possible. In this context, very rapid heating implies a temperature increase in the article of food in the order of 1° C. per second. It is also desirable to use heating methods that render possible to use of a conveyor belt, in which the food is located on the conveyor belt before, during and after the heat treatment. Trials have shown that the above can be achieved at least by using radio waves and by heat-treating the product while being submerged in a bath of liquid.

In order to provide precooked food products of the highest possible quality, extensive research is done on processing methods for preserving the properties of the product in the best possible way. In this research, some tests are known to provide “quality indicators” with respect to how the different processing methods affect the properties of the food, among other things. The most central of these tests concern the measuring of texture, water or liquid retention ability, cooking loss and colour changes during processing. In the art, the water retention ability is referred to as WHC, i.e. water holding capacity, and the liquid retention ability is referred to as LHC, i.e. liquid holding capacity. The purpose of said tests, as well as what the results thereof indicate, are well known to a person skilled in the art and hence will not be explained in further detail in this document.

With respect to the utility value of the above-mentioned tests, it is of course imperative that the processing of the test pieces in a laboratory, for example, is approximately identical to the industrial processing sought tested.

Likewise, it is desirable to be able to provide a test cup that is arranged so as to be able to simulate the heat treatment that a consumer normally exposes for example raw meat to, and then carry out measurements of one or more of the above-mentioned quality indicators in order to provide measurement results for the meat whilst in the condition available to the consumer.

Different types of test devices are known, which are adapted for individual measurements of the above-mentioned quality indicators for an article of food, such as raw meat. A test cup, for example, is known to be used when centrifuging for the purpose of testing the water or liquid holding capacity of the meat. The test cup is produced from a relatively thick-walled plastics material, in which the test cup is comprised of a cylindrical element wherein a filter is placed so as to divide the test cup into a bottom portion and top portion. The meat to be tested is placed onto the filter. A bottom lid is placed in the bottom portion, thereby allowing the water or liquid released from the meat by means of centrifugation to be collected in a container defined by the bottom lid and the cylinder wall.

There are several disadvantages related to the above-mentioned prior art.

The prior art test cup is not suitable for use in heat treatment simulation by means of radio waves or other type of heating, in which the article of food is submerged in a liquid during the heat treatment. Firstly, this is due to the test cup not being liquid-tight, thereby allowing liquid to enter into the test cup, or thereby allowing liquid from the meat, for example, to evaporate out of the test cup. Secondly, the thermal conductivity of the test cup is insufficient, thereby possibly allowing the test piece to undergo a relatively slow heating. In addition, it is only possible for the meat to bear against the sides of the test cup and not to bear against the bottom portion and possibly the top portion thereof. Consequently, the heat supply through the bottom and top will be greatly limited, insofar as insulating air will prevent an efficient heat supply to the meat. If the prior art test cup were to be used for heat treatment, the heating time for the meat would be unfavourably long, in the order of 1° C. per minute. Instead, the meat therefore must be heat-treated separately from the test cup and then be transferred to the prior art test cup. This is difficult to carry out on meat consisting, for example, of whole pieces of fish, insofar as boiled fish meat readily disintegrates when handled. Additionally, the handling may affect the result of the subsequent measuring of water or liquid holding capacity. Moreover, when testing texture, for example, the meat must undergo yet another transfer from the test cup, insofar as the meat must be removed from the filter and be placed on a firm base that does not yield when subjected to the compressive force of the measuring apparatus against the meat.

Patent documents EP 0297415 B1, DE 4406658 A1 and SE 0385737 B disclose known systems and/or methods for treating biological material, in which the biological material is treated in a tubular test container. None of the systems and methods of said patent documents includes the possibility of providing one or more removable and sealing bottom lids, and one or more removable and sealing top lids. Therefore, the tubular test containers of said patent documents are not suitable for use in accordance with the object of the present invention.

The object of the invention is to remedy or at least reduce one or more disadvantages of the prior art.

The object is achieved through features disclosed in the description below and in the subsequent claims.

Positional references in this document, such as “upper” and “lower”, “bottom” and “top” or “horizontal” and “vertical”, denote the position of the test cup as viewed in the attached drawings, the position which is also refer to a natural, required or practical position of use.

In one aspect, the present invention comprises a test cup for use when testing articles of food, such as food and feeds. In a preferred embodiment, the test cup is comprised of a tubular body arranged for internal reception of a filter. In a preferred embodiment, the filter is comprised of a filter element fixedly attached to a surrounding peripheral supporting element. The filter divides the internal cavity of the test cup into a bottom portion and a top portion, within which the test piece is placed. In one configuration, the test cup is provided with one of at least two dissimilar, removable and sealing bottom lids, and also with one of at least two dissimilar, removable and sealing top lids. Moreover, this configuration is adapted for the type of testing to be carried out.

In a preferred embodiment, the filter is arranged in a manner allowing selective division of the size-ratio between said bottom portion and said top portion. In one embodiment, said selective division is achieved by attaching the filter to the internal portion of the tubular body by means of a threaded connection, whereby the size of the top portion may be adapted to the thickness of the test piece placed within the test cup. Other connecting means known per se may also be used instead of the threaded connections. Although a test cup of a cylindrical shape is preferred, other shapes are not to be excluded. For example, a test cup having a triangular, rectangular or polygonal shape, as viewed in horizontal section, may also be used.

In order to obtain a good heat transfer when heat-treating the test piece, it is very important for the test piece to bear as much as possible against a top lid or a bottom lid in addition to bearing against the side(s) of the test cup.

In order to obtain a correct measurement when testing the texture of the test piece, it is imperative for the test piece to bear against a firm base. The test cup according to the present invention is therefore arranged so as to be able to receive a first bottom lid provided with a raised bottom portion projecting towards the filter, said bottom portion preferably being complementary with the filter element of the filter. Thereby, said raised bottom portion will bear against the filter element when a peripheral portion of the bottom lid bears against the supporting element of the filter.

In order to collect the liquid released during tests for water or liquid holding capacity, there is a need for a cavity arranged on the opposite side of the filter upon which the test piece is placed. The test cup is therefore arranged so as to be able to receive a second bottom lid defining a cavity below the filter.

In a preferred embodiment, said bottom lid is attached to the internal portion of the tubular body by means of a threaded connection.

In a preferred embodiment, and in order to ensure that no fluid migration takes place between the test cup's internal portion and the outside thereof, or vice versa, the lid of the test cup is provided with a sealing means. In a preferred embodiment, the sealing means comprises one or more O-rings known per se. Moreover, the sealing surface of the top lid may also be comprised of an imperforated body.

In one embodiment, the top lid is provided with a bushing arranged so as to be able to provide a passage for a probe, for example, or other elongated body extending between the test cup's internal portion and the outside thereof. For example, the probe may be a device for measuring of temperature(s) in the food. In a preferred embodiment, the bushing is provided with a sealing means for providing a seal between the bushing and the probe.

In a preferred embodiment, and in order to ensure a good heat transfer from the heated liquid, into which the test cup is submerged, to the test piece, the test cup is produced from a rigid, thin-walled material of good thermal conductivity. Good thermal conductivity in this context is taken to imply materials having a thermal conductivity of approximately 30 W/(m*K) and higher. As such, at least a portion of the test cup is produced from metal. In tests wherein a high thermal conductivity is of subordinate importance, materials of lower thermal conductivity may also be used for producing all or portions of the test cup.

In another aspect, the invention concerns a method of using a test cup when testing an article of food including, but not limited to, food, feed or meat, in which the test cup is comprised of a tubular body arranged for internal reception of a filter that divides the internal cavity of the test cup into a bottom portion and a top portion. The method also comprises the provision of one or more removable and sealing bottom lids, and also one or more removable and sealing top lids. The test cup is configured with a bottom lid and a top lid that are adapted for the type of testing to be carried out. Preferably, the configuration thereof is carried out without the test piece, which is being tested, having to be touched or removed from the test cup. In order to provide as good a heat transfer as possible between the outside of the test cup and the test piece of food, the test piece is adapted to the internal surface of the tubular body, and the top lid and the bottom lid are arranged to bear against substantially the top side and bottom side of the test piece.

Hereinafter, a non-limiting example of a preferred embodiment is described and visualized in the accompanying drawings, in which:

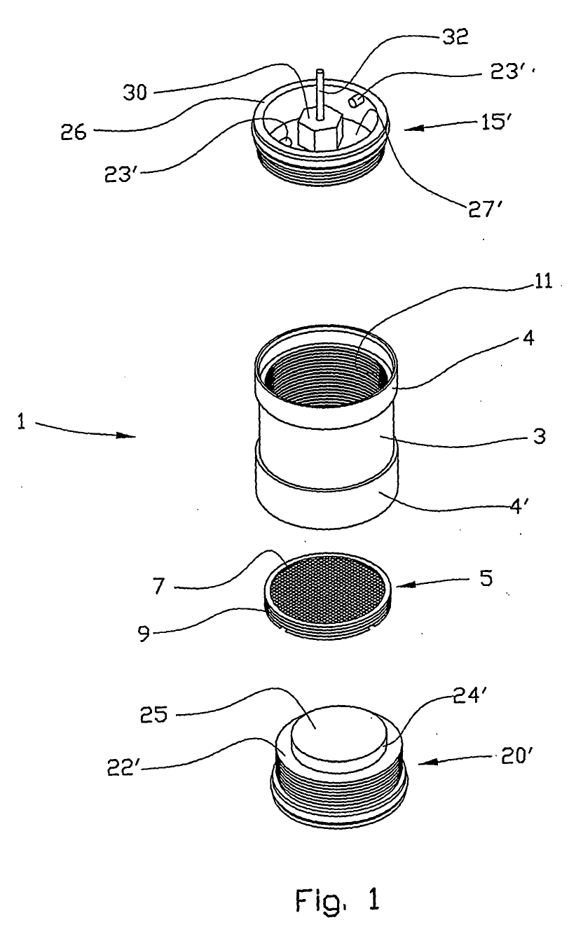

FIG. 1 shows a perspective front view of a test cup according to the invention, in which the components of the test cup are shown separated from each other.

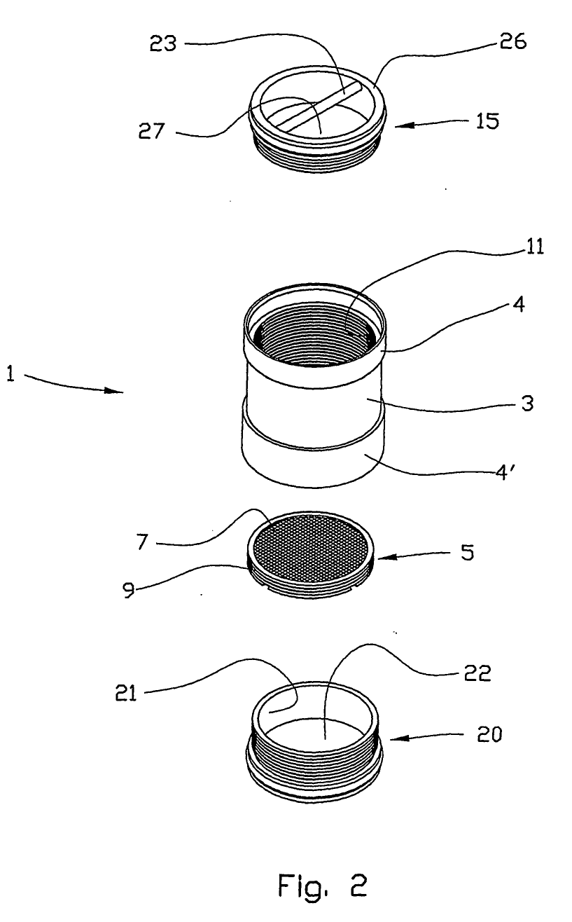

FIG. 2 shows a perspective front view of the test cup of FIG. 1, although shown with an alternative shape of the test cup's top lid and bottom lid.

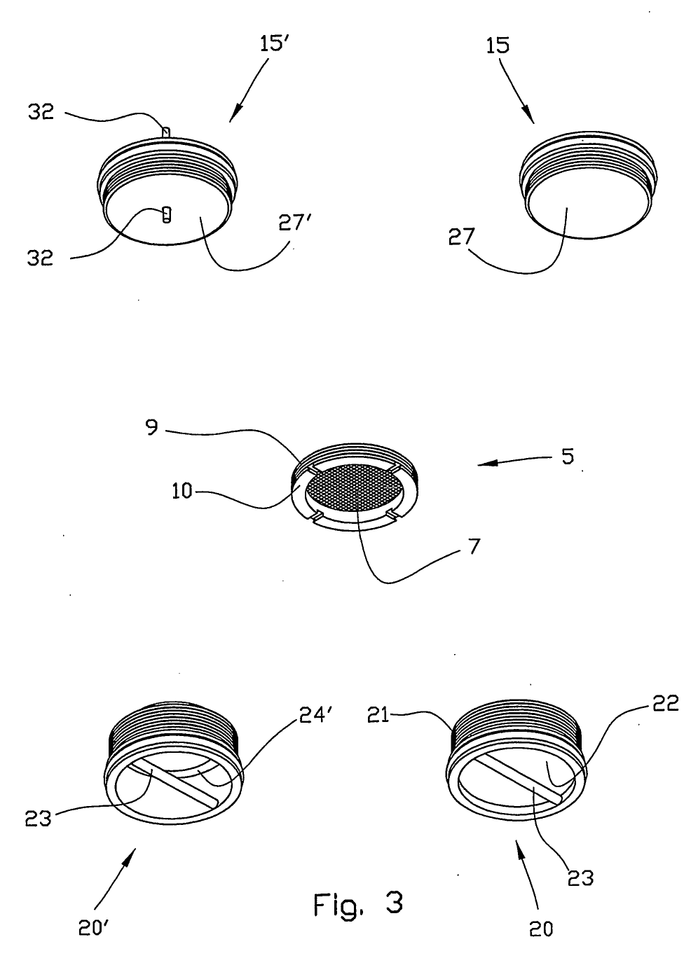

FIG. 3 shows a front view of the test cup's top lid, bottom lid and filter shown in FIGS. 1 and 2, although viewed in perspective from below.

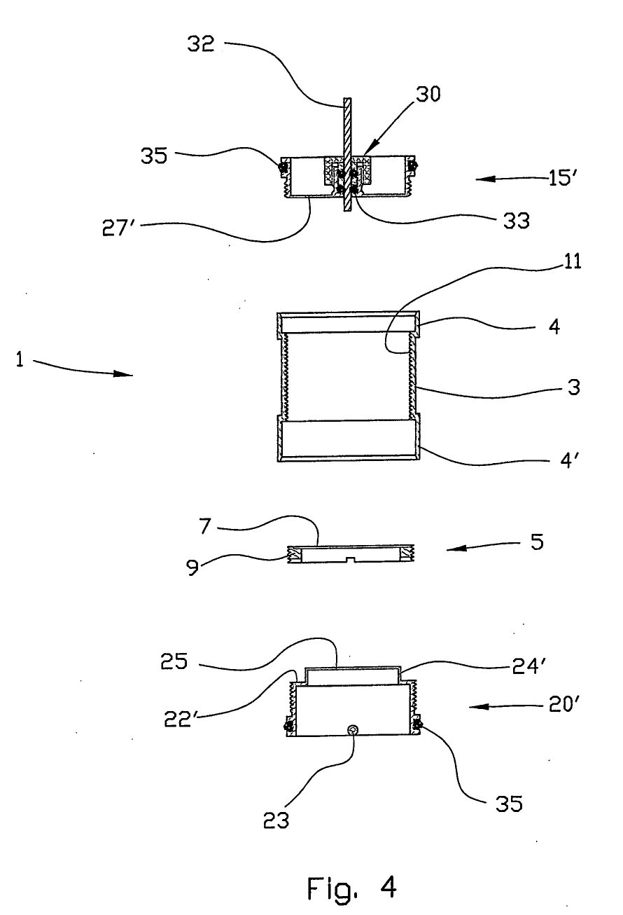

FIG. 4 shows a vertical section through the centre axis of FIG. 1.

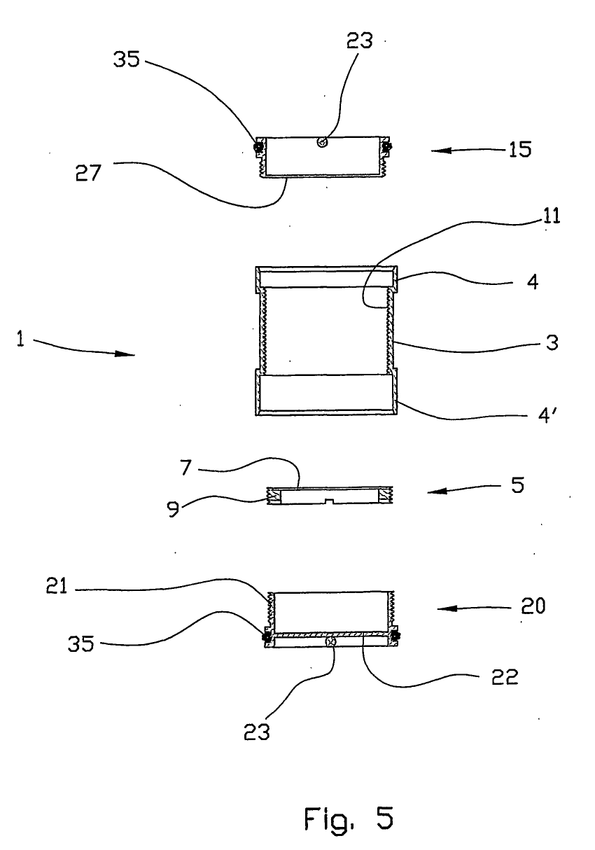

FIG. 5 shows a vertical section through the centre axis of FIG. 2.

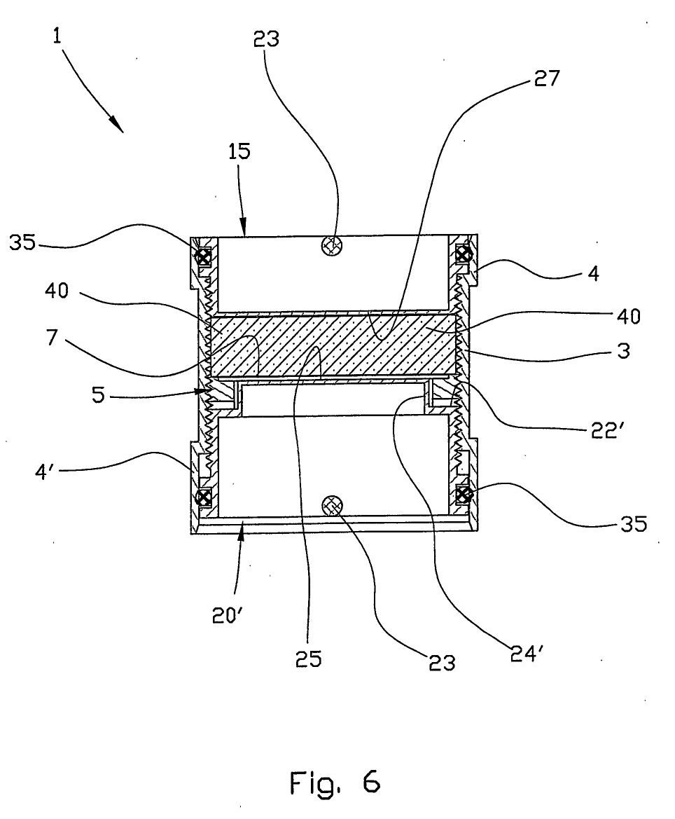

FIG. 6 shows, in larger scale, a vertical section through the centre axis of a test cup whilst having a piece of test material placed therein.

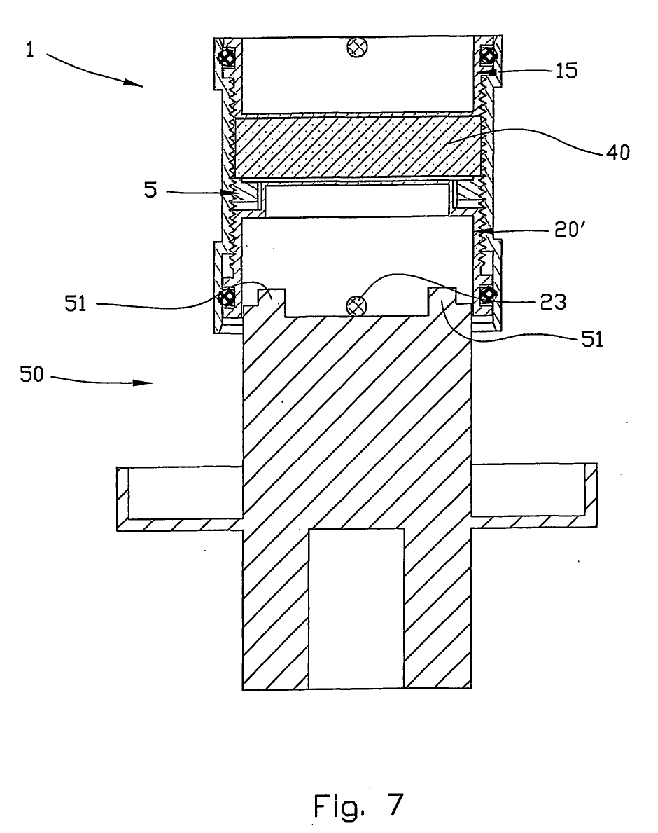

FIG. 7 shows the test cup of FIG. 6 whilst a screw-type tool is engaged with the bottom lid.

In the drawings, the reference numeral 1 denotes a test cup comprised of a cylindrical tubular body 3 provided with an upper rim 4 at the upper end portion thereof, and provided with a lower rim 4′ at the lower end portion thereof. The cylindrical tubular body 3 is arranged so as to be able to receive a filter 5, which is comprised of a filter cloth 7 fixedly attached to a filter ring 9. At its vertical and external end surface, the ring 9 of the filter 5 is provided with threads arranged so as to be able to mate with complementary threads formed in the internal wall surface 11 of the tubular body 3. Thus, the filter 5 may be placed in any suitable portion of the tubular body 3, and thereby the positioning of the filter 5 may be adapted to the size of the test piece.

Further, the test cup 1 is comprised of a top lid 15, 15′ and a bottom lid 20, 20′. The top lid 15, 15′ is placed onto a top portion of the cylindrical tubular body 3, at the upper rim 4 thereof, whereas the bottom lid 20, 20′ is placed onto a bottom portion of said cylindrical body 3, at the lower rim 4′ thereof.

In order to ensure that the lids 15, 15′, 20 and 20′ form a tight connection with the tubular body 3, the lids are provided with sealing means, which comprises O-rings 35 known per se, and which are best shown in FIGS. 4 and 5.

FIG. 1 shows a test cup 1 in a configuration prior to assembly with a first top lid 15′ and a first bottom lid 20′. The first top lid 15′ is provided with a bushing 30 of a type known per se, which is placed centrally on the sealing surface 27′, and which is connected to an opening through the sealing surface 27′. A probe 32 for measuring temperature, for example, is passed through the bushing 30 and sealing surface 27′. The bushing 30 is provided with a sealing means 33, which seals between the probe 32 and the wall portion of the opening or channel. The first bottom lid 20′ is provided with a cylindrical stub 24′ projecting coaxially upwards from a bottom surface 22′ of the bottom lid 20′. The stub 24′ has an external diameter adapted to the internal diameter of the filter ring 9 to allow the stub 24′ to be inserted into the filter ring 9. The purpose of the stub 24′ is both to provide a support for the filter cloth 7, thereby avoiding deflection thereof in connection with the measuring of the test piece texture, and also to provide maximum contact between the test piece and the bottom lid 20′. The stub 24′ is adapted to the filter 5 so as to allow the height of the stub 24′ above the bottom surface 22′ to substantially correspond to the distance between the lower end surface 10 of the filter ring 9 (see FIG. 3) and the underside of the filter cloth 7. This renders possible for the top surface 25 of the stub 24′ thus to bear against the underside of the filter cloth 7, and simultaneously allowing the bottom surface 22′ of the first bottom lid 20′ to bear against the end surface 10 of the filter ring 9. The first bottom lid 20′ is provided with a gripping element in the form of a rod 23.

FIG. 2 shows the test cup 1 provided with the same cylindrical tubular body 3 and filter 5 as shown in FIG. 1, but provided with an alternative second top lid 15 and an alternative second bottom lid 20. The second bottom lid 20 is comprised of a cylinder portion 21 projecting upwards from a bottom portion 22. On its external surface, the cylinder portion 21 is provided with threads arranged so as to be able to mate with complementary threads formed in the internal wall surface 11 of the tubular body 3. In the embodiment shown (see FIG. 3), the bottom surface 22 is placed somewhat into the cylinder portion 21, whereby a cavity is formed. A gripping element 23 corresponding to the gripping element 23 in the first bottom lid 20′, is placed in this cavity. The top lid 15 is comprised of a cylindrical element 26 projecting upwards from a sealing surface 27.

In heat treatment in test laboratories, for example, and when the fastest heating of the test piece is desired, for example in simulation of heat treatment by means of radio waves, it is imperative that the test piece has maximum contact with the surfaces of the test cup 1, thereby avoiding insulating air pockets as much as possible. The test cup 1 according to the present invention is arranged so as to be able to be configured for optimal heat conduction between the external surface of the test cup 1 and the test piece to be heat-treated. FIG. 6 shows a vertical section through a test cup 1, in which a test piece 40 is placed on the filter 5. The test piece 40 may, for example, be a piece of meat. In the assembly shown, the test cup 1 is provided with a second top lid 15 and a first bottom lid 20′, in which the top surface 25 of the stub 24′ of the bottom lid 20′ bears against the underside of the filter cloth 7. The test piece 40 is substantially in contact with the wall 11 of the tubular body 3, and simultaneously the sealing surface 27 of the top lid 15 bears against the upper surface of the test piece 40. Accurate adaptation of the test piece with respect to the test cup 1 is achieved by using of a punching tool (not shown), known per se, having an internal diameter substantially corresponding to the internal diameter of the tubular body 3. Trials have been carried out with prototypes of the test cup 1. The test cup 1 was produced from stainless steel, in which the sealing surface 27, 27′ of the top lid 15, 15′ and also the top surface 25 of the first bottom lid 20′ were produced having walls of a 0.5 mm thickness. Moreover, the tubular body 3 of an internal diameter of 31 mm, was produced having a wall of approximately 1.5 mm thickness. The noted trials showed that the rate of temperature increase in the test piece 40 substantially corresponded to the rate of temperature increase achieved by means of radio waves.

When carrying out measurements of, for example, texture, liquid holding capacity, cooking loss and colour change after heat treatment using the test cup 1 according to the present invention, it is entirely possible to carry out the desired measurements without having to touch or move the test material placed on the filter 5. Among other things, this is very important for meat of for example fish in whole pieces, insofar as the pieces readily may be destroyed, and hence become unsuitable for the desired measurements, when touched or moved. As already mentioned above, said first bottom lid 20′ is used in connection with heat treatment and/or measuring of texture. When measuring the liquid holding capacity, the first bottom lid 20′ is replaced with the second bottom lid 20, thereby providing a cavity for receiving the liquid released from the test piece 40 and passed through the filter cloth 7 during, for example, a centrifugation process known per se. The test cup configuration to be used for the various measurements, will be obvious to a person skilled in the art.

Upon using the present test cup 1 for measuring, for example, cooking loss or liquid holding capacity, relatively small test pieces are used, typically of a weight in the order of 5-10 g. The liquid amounts released from the test piece 40 are therefore relatively small. For this reason, it is important that as much as possible of the liquid released from the test piece 40 is removed from the internal surface of the test cup 1. Advantageously, at least a portion of the internal surface of the test cup 1 are provided with a heat-resistant, liquid-repellent coating to facilitate the removal of liquid therefrom. Such a coating may be comprised of, but not limited to, materials sold under the trade mark Teflon.

In order to facilitate the screwing in and out of, for example, the top- and bottom lids of the test cup, a screwing tool 50 is provided, the tool of which is arranged so as to be able to engage the gripping device 23 of the lids 15, 15′, 20, 20′. FIG. 7 shows a vertical section of a screwing tool 50 inserted into a bottom portion of the bottom lid 20′. Upon subjecting the screwing tool 50 to a relative rotation with respect to the test cup 1, the pegs 52 of the screwing tool 50 will engage and bear against the gripping device 23, whereby the bottom lid 20′ is screwed into or out of the tubular body 3. The screwing tool 50 may also be used for screwing the top lid 15, 15′ in and out of the tubular body 3. The gripping device placed in the first top lid 15′ is comprised of two pins 23′ projecting from an internal side portion of the lid 15′. In a preferred embodiment, the screwing device 50 is attached to an electric drilling machine (not shown) of a type known per se.

Although described hereinbefore substantially for heat treatment use and subsequent measuring of the test piece, it is to be understood that the test cup also is suitable for measurements performed on food and feeds which that are not heat-treated, and also on chopped-up food and feeds that hence are not in whole pieces.

1. A test cup (1) for use in testing an article of food (40), such as food and feed, in which the test cup (1) is comprised of a tubular body (3) arranged for internal reception of a filter (5) that divides an internal cavity of the test cup (1) into a bottom portion and a top portion, wherein the test cup (1) is provided with one of at least two dissimilar, removable and sealing bottom lids (20, 20′), and also with one of at least two dissimilar, removable and sealing top lids (15, 15′), the configuration of which is adapted for the type of measurement to be carried out.

2. The test cup (1) according to claim 1, wherein the filter (5) is arranged in a manner allowing selective division of said bottom portion and said top portion.

3. The test cup (1) according to claim 2, wherein the filter (5) is attached to an internal portion (11) of the tubular body (3) by means of a threaded connection.

4. The test cup (1) according to claim 1, wherein a second bottom lid (20) is comprised of a cylinder portion (21) projecting upwards from a bottom portion (22).

5. The test cup (1) according to claim 1, wherein a first bottom lid (20′) is provided with a cylindrical stub (24′) projecting upwards from a bottom portion (22′) of the bottom lid (20′), wherein the stub (24′) is complementary with the filter ring (9) of the filter (5) in a manner allowing a top surface (25) of the stub (24′) to bear against the underside of a filter cloth (7), and simultaneously allowing a peripheral portion of a bottom surface (22′) of the bottom lid (20′) to bear against an end surface (10) of the filter ring (9) facing the bottom lid (20′).

6. The test cup (1) according to claim 4, wherein the bottom lid (20, 20′) is attached to the internal portion (11) of the tubular body (3) by means of a threaded connection.

7. The test cup (1) according to claim 1, wherein a sealing surface (27) of the top lid (15) is comprised of an imperforated body.

8. The test cup (1) according to claim 1, wherein the top lid (15′) is provided with a bushing (30) arranged so as to be able to receive and provide a seal around a probe (32) inserted through a sealing surface (27′) of the top lid (15′).

9. The test cup (1) according to claim 7, wherein the top lid (15, 15′) is attached to the internal portion (11) of the tubular body (3) by means of a threaded connection.

10. The test cup (1) according to claim 1, wherein at least a portion of the test cup (1) is produced from a thin-walled material of good thermal conductivity.

11. The test cup (1) according to claim 10, wherein at least a portion of the test cup (1) is produced from metal.

12. The test cup (1) according to claim 1, wherein the top lid (15, 15′) and bottom lid (20, 20′) are provided with at least one gripping element (23, 23′).

13. The test cup (1) according to claim 12, wherein a rotatable screwing device (50) is arranged so as to be able to engage the at least one gripping element (23, 23′) of the lid (15, 15′, 20, 20′).

14. The test cup (1) according to claim 1, wherein at least a portion of the internal surface of the test cup (1) is provided with a heat-resistant, liquid-repellent coating.

15. A method of using a test cup (1) when testing an article of food (40), such as food or feed, in which the test cup (1) is comprised of a tubular body (3) arranged for internal reception of a filter (5) that divides an internal cavity of the test cup (1) into a bottom portion and a top portion, wherein the method comprises the provision of one or more removable and sealing bottom lids (20, 20′), and also one or more removable and sealing top lids (15, 15′), wherein the test cup (1) is configured with a bottom lid (20, 20′) and a top lid (15, 15′) that are adapted for the type of testing to be carried out.

16. The method according to claim 15, wherein a bottom surface (21, 21′) of the top lid (15, 15′) is arranged to bear against substantially the entire upper portion of the test piece (40).

17. The method according to claim 15, wherein a top surface (25) of a stub (24′) of the first bottom lid (20′) is arranged to bear against the filter (5).

18. The method according to claim 15, wherein the extent of the test piece (40) is adapted in a manner allowing a side portion of the test piece (40) to bear against a portion of an internal surface (11) of the tubular body (3).

19. The method according to claim 15, wherein the test cup (1) is configured to the type of testing to be carried out, and without the test piece (40), which is being tested, having to be touched or removed from the filter (5) of the test cup (1).
